(19) United States
(12) Patent Application Publication
(10) Pub. No.: US 2024/0237950 A1
(43) Pub. Date: Jul. 18, 2024

(54) METHOD FOR ASSIGNING AN ITEM OF IDENTIFICATION INFORMATION TO A SIGNAL FROM A CARDIAC ELECTRODE

(71) Applicants: CENTRE HOSPITALIER UNIVERSITAIRE DE BORDEAUX, TALENCE (FR); UNIVERSITE DE BORDEAUX, BORDEAUX (FR); FONDATION BORDEAUX UNIVERSITÉ, BORDEAUX (FR)

(72) Inventors: Sylvain PLOUX, GRADIGNAN (FR); Marc STRIK, BORDEAUX (FR); Pierre BORDACHAR, BORDEAUX (FR); Rémi DUBOIS, MÉRIGNAC (FR)

(21) Appl. No.: 18/557,123

(22) PCT Filed: Apr. 27, 2022
(86) PCT No.: PCT/EP2022/061247

§ 371 Date: Oct. 25, 2023

(30) Foreign Application Priority Data
Apr. 27, 2021 (FR) FR2104376
Nov. 17, 2021 (FR) FR2112163

(51) Int. Cl. A61B 5/00; A61B 5/318

(52) U.S. Cl. CPC A61B 5/7221; A61B 5/318; A61B 5/746

(57) ABSTRACT

A computer implemented method for assigning an item of identification information to a first detection signal includes acquiring a detection signal in response to receiving a cardiac electrical current, the detection signal including a signal portion corresponding to an electrical pattern over a first interval; implementing a comparison algorithm including comparing the first interval with a first threshold; comparing a second interval associated with a portion of a second detection signal prior to the first detection signal with a second threshold; assigning an item of identification information to the first detection signal, the item of identification information being determined based on the result of the comparisons performed by the comparison algorithm.

### FIELD OF THE INVENTION

The field of the invention relates to the field of methods and devices for detecting, analyzing, and processing cardiac signals. In particular, the field of the invention relates to the field of methods for identifying characteristics of cardiac signals from one or more detection probes, such as one or more cardiac electrodes, and for assigning information to said signals.

### PRIOR ART

In the prior art, there are methods applied to cardiac pacemakers also known in literature as “pacemaker” or “cardiac battery”, and to defibrillators such as automatic implantable cardiac defibrillators, also known in literature as “ICD”, to deliver therapeutic shocks to patients.

Such devices comprise electrodes, also known as probes, to detect the heart rate and even to deliver “therapeutic” stimulations and/or electrical shocks to patients. When a change in the rate is detected, such as a characteristic rhythm of bradycardia, ventricular fibrillation, or ventricular tachycardia, therapeutic electrical stimulation or shock may be delivered by the device to restore a natural heart rate.

However, there is a problem when a pacemaker or defibrillator probe is damaged. When a probe is damaged on a “pacemaker”, there is a risk of the heart of the patient stopping and therefore a risk of death. A damaged probe may be caused, for example, by a broken internal cable of the probe or by open insulation.

In both cases, this is indicated by the appearance of noise, intermittently or continuously, on a detection channel of the probe. However, noise appearing on the detection channel may be misinterpreted as a signal of cardiac origin. This signal will have the consequence of inhibiting stimulation on a pacemaker with a risk of cardiac arrest and/or leading to the delivery of an electrical shock, even repeated electrical shocks, by a defibrillator. These inappropriately delivered electrical shocks may have dramatic consequences for patients. Therefore, a method should be put in place to distinguish pathological signals from signals due to a defective electrode.

There are, in the prior art, methods that make it possible to overcome this issue of inappropriately delivered electrical shocks. For example, U.S. Pat. No. 7,369,893 describes a method for evaluating whether the detection of a signal by a pacemaker probe is indeed related to a cardiac arrhythmia phenomenon and not to an overdetection phenomenon, by detecting a condition linked to the probe. This is because it is possible that an electrode is defective due to a broken conductor and results in the appearance of noise on the detection channel. However, such a method has the disadvantage of not being accurate in detecting noise, the appearance of which may be due to multiple factors.

The invention aims to exploit another way of determining the presence of noise, in particular by directly interpreting the signals on a detection channel of a probe. Indeed, on the basis of the proven principle that the heart of a human being cannot contract several times in an excessively short time interval, it is possible to observe on the detection channel of an electrode that a signal cannot be physiological; by comparing the time intervals separating several cardiac cycles. Therefore, the appearance of a non-physiological signal on the detection channel may be attributed to noise, and consequently, to a high probability that an electrode is defective.

An objective of the invention is to solve the disadvantages due to the appearance of noise on a detection channel of an electrode by proposing a computer implemented method to assign an item of identification information to a signal from the detection of a cardiac electrical current. The method is based on time intervals separating several detected signals, such as time intervals between several successive signals, and advantageously makes it possible to accurately detect the appearance of noise on a detection channel. Another advantage is to reduce the number of alerts emitted by defibrillators and “pacemakers” in response to the detection of false positives of ventricular arrhythmias.

### SUMMARY OF THE INVENTION

According to a first aspect, the invention relates to a computer implemented method for assigning an item of identification information to a detection signal, characterized in that it comprises:
acquiring, by means of a probe, a first detection signal in response to receiving a cardiac electrical current, said first detection signal comprising a signal portion corresponding to an electrical pattern over a first interval;
applying a comparison algorithm comprising:
   comparing the first interval with a first threshold;
   comparing a second interval associated with a portion of a second detection signal prior to the first detection signal with a second threshold;
assigning an item of identification information to the first detection signal, said item of identification information being determined based on the result of comparisons made by the comparison algorithm, said item of identification information making it possible to discriminate a detection signal having a physiological cause from a detection signal having as its cause a malfunction of electronic hardware.

One advantage is to determine, based on the results of the comparisons applied by the algorithm, whether a detected signal actually corresponds to a physiological signal; or whether it is a measurement artifact potentially due to a defective cardiac probe. Another advantage is to detect the presence of noise by applying a low-complexity algorithm, which may therefore be implemented on devices with little computing power.

According to one embodiment, the comparison algorithm comprises a step of comparing a third interval associated with a portion of a third detection signal with a third threshold, said third detection signal being prior to the second detection signal.

One advantage is to improve noise identification on the detected signals by adding a comparison step in the algorithm.

According to one embodiment, the method comprises implementing a second comparison algorithm comprising:
A step of comparing the first interval with a fourth threshold;
A step of comparing the second interval with a fifth threshold;
A step of comparing the third interval with a sixth threshold.

One advantage is to improve noise detection on a detection channel of a probe; by implementing another algorithm comprising additional comparison steps.

According to one embodiment, the method comprises implementing, by the first comparison algorithm or the second comparison algorithm, the comparison of a seventh threshold with a fourth time interval associated with a portion of a fourth detection signal prior to the third detection signal.

One advantage is that it is possible to distinguish between signals characteristic of overdetection phenomena and signals characteristic of a break in the probe.

According to one embodiment, one or more thresholds among the first, second, third, fourth, fifth, sixth or seventh thresholds are defined by:
A predefined threshold value or;
A median value or an average of several values together or;
A mathematical function defined in relation to one or more time intervals or;
A mathematical function independent of the values of the acquired intervals or;
A combination of several mathematical functions.

One advantage is to define the criteria for assigning the type of item of identification information from more complex thresholds to obtain additional precision on noise detection on the detection channel.

According to one embodiment, the item of identification information is assigned to the first detection signal according to the result of comparisons of at least one of the two comparison algorithms.

One advantage is to use the results of comparisons of at least one of the algorithms to accurately identify the signal on the detection channel of the electrode.

According to one embodiment, the item of identification information assigned to the first detection signal comprises either:
an item of physiological information defining a natural heartbeat or cardiac arrhythmia;
an item of anomaly information characterizing an item of non-physiological signal data.

One advantage is to characterize the item of identification information assigned to the detection signal to determine whether said detection signal corresponds to the detection of a heart rate or whether the detection signal corresponds to a measurement artifact, for example due to a malfunction of an electrode.

According to one embodiment, the method comprises:
Generating a notification or alert when the item of identification information assigned to the first detection signal comprises an item of anomaly information;
Recording said alert/notification in a memory space,
Emitting said alert/notification generated to equipment in a remote data network.

One advantage is to alert skilled staff when a defect on an electrode of a patient is found.

According to one embodiment, the item of identification information assigned to the first detection signal comprises an item of anomaly information either when:
The first interval is less than the first threshold and the second interval is greater than the second threshold;
The first interval is less than the first threshold and the second interval is greater than the second threshold and the third interval is greater than the third threshold.

One advantage is to deduce the presence of a non-physiological signal on the detection channel, based on comparison results between characteristic threshold time values and the intervals associated with the detection signals.

According to one embodiment, the method comprises:
generating an electrogram, said electrogram comprising a graphical representation of at least one detection signal among the signals and of at least one interval;
acquiring at least one image of the electrogram;
recording the image of the electrogram acquired in a memory space;
transmitting the image of the acquired electrogram to at least one piece of equipment of a data network.

One advantage is to be able to record and transmit to remote equipment a graphic representation of the signals detected, which may subsequently be examined by skilled personnel to confirm or invalidate the presence of noise on the detection channel.

According to one embodiment, the method comprises generating a visual, audible and/or vibration alert when the item of identification information comprises an item of anomaly information.

One advantage is to alert the patient to the presence of an item of anomaly potentially due to the malfunction of a piece of equipment.

According to one embodiment, the item of identification information assigned to the first detection signal comprises an item of anomaly information characteristic of a probe breakage:
Either when the first time interval is less than the first threshold, the second time interval is greater than the second threshold and the third time interval is greater than the third threshold;
Or when the first time interval is less than the first threshold and the second time interval is greater than the second threshold, the third time interval is greater than the third threshold and the fourth time interval is greater than the seventh threshold.

According to another aspect, the invention relates to a system for generating an item of identification information of a cardiac detection signal comprising:
An electrical device comprising at least one probe for acquiring a first detection signal in response to the reception of a cardiac electrical current, said detection signal comprising a signal portion corresponding to an electrical pattern over a first interval;
A calculator configured to implement:
   on the one hand a comparison algorithm comprising:
      i. Comparing the first interval with a first threshold;
      ii. Comparing a second interval associated with a portion of a second detection signal prior to the first detection signal with a second threshold;
on the other hand, a function for assigning an item of identification information to the first detection signal, said item of identification information being determined according to the result of comparisons made by the comparison algorithm, said item of identification information making it possible to discriminate a detection signal having a physiological cause from a detection signal having a malfunction of electronic hardware as its cause.
A display for generating a graphic marker overlaying an electrogram comprising the first detection signal in order to temporally locate the item of identification information of the first detection signal.
A memory to record data;
A communication interface to exchange data with equipment in a data network.

According to another aspect, the invention relates to a system configured to implement any one of the steps of the method according to the invention, said system comprising:
the electrical device comprising at least one probe for receiving a cardiac electrical current;
a signal generator to generate the detection signal in response to the receiving of the cardiac electrical current;
a memory to record data;
a communication interface to exchange data with equipment in a remote data network;
According to one aspect, the invention relates to a computer program product comprising instructions which, when the program is executed by a computer, lead the latter to implement the following steps
Implementation of a first comparison algorithm comprising:
   Comparing a first time interval, over which an electrical pattern extends from a portion of a first detection signal acquired by means of a probe, with a first threshold;
   Comparing a second time interval associated with a portion of a second detection signal prior to the first detection signal with a second threshold;
Assigning an item of identification information to the first detection signal, said item of identification information being determined according to the result of comparisons made by the comparison algorithm and said item of identification information making it possible to discriminate a detection signal having a physiological cause from a detection signal having as its cause a malfunction of electronic hardware.

### BRIEF DESCRIPTION OF THE FIGURES

Other characteristics and advantages of the invention will become clearer upon reading the following detailed description, in reference to the appended figures, that illustrate:

FIG. 1: An electrical device comprising a probe implemented in the heart of a patient.

FIG. 2: A flowchart of the implementation of the comparison algorithm by a calculator to obtain the item of identification information.

FIG. 3: A flowchart of the comparison algorithm implemented by a calculator when it comprises two comparison steps.

FIG. 4: A flowchart of the comparison algorithm implemented by a calculator when it comprises three comparison steps.

FIG. 5: A flowchart of the implementation of two comparison algorithms comprising a plurality of comparison steps.

FIG. 6: A visual of an electrogram comprising a graphical representation of three detection signals and associated intervals.

FIG. 7: A flowchart of the transmission of an alert to a piece of equipment of a data network.

FIG. 8: An example of a threshold defined by a function of two successive time intervals.

FIG. 9: a flowchart of the implementation of two comparison algorithms comprising a plurality of comparison steps.

FIG. 10: A visual of an electrogram comprising a graphical representation of four detection signals, and associated intervals.

### DETAILED DESCRIPTION

According to a first aspect, the invention relates to a computer implemented method to assign an item of identification information I_{id} to a first detection signal S_{D}.

### Detection Signals

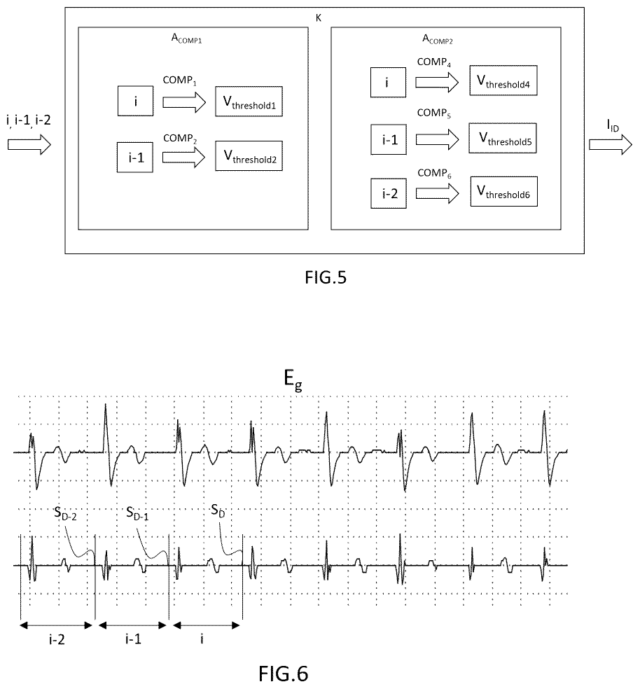

It should be understood by “detection signal” a signal portion characteristic of either cardiac electrical activity or of a hardware defect, such as a break in a probe, or of one or more external signals such as electromagnetic interference. In an illustrative example, in reference to FIG. 6, an image of an electrogram E_{G} comprises a graphical representation of several detection signals S_{D}, S_{D-1}, S_{D-2} each representing a portion of cardiac electrical activity as a function of time.

In the remainder of the description, a time interval is called the time during which a detection signal S_{D} is defined and acquired. Thus, the first detection signal S_{D} is associated with a first interval i. The first interval i corresponds to a time interval. The time interval i is defined between two successive events. Events are, for example, patterns or recognizable electrical signatures or characteristics of a physiological or non-physiological cause.

The notion of time interval i is therefore linked to the sequential sequence of measurements of an electrical signal of a probe positioned in the vicinity of an electrical source producing a regular signal such as the heart.

The events may correspond to singularities of an acquired electrical pattern such as:
A local maximum;
A local minimum;
The duration or the presence of two local maximums or local minimums or a combination of one local maximum and one local minimum;
A given frequency or a given spectral pattern, more generally a spectrum;
Etc.

According to one embodiment, the events are subsequently identified from the acquisition of a detection signal S_{D}, for example from at least two signals S_{D-1} and S_{D} acquired to deduce therefrom a period or pseudo-period between two comparable portions of two successively acquired detection signals. This solution makes it possible to guarantee that reliable events may be considered to extract an interval calculation therefrom.

More generally, an event is defined to constitute an interval measurement reference.

In one example, the first interval i comprises the time interval separating the first detection signal S_{D} from another prior signal, for example the second detection signal S_{D-1}. It should be understood by “prior” that the second detection signal S_{D-1} was detected by the electrode before the first detection signal S_{D}. For example, the two signals S_{D} and S_{D-1} are detected successively. From a physiological point of view, the interval i corresponds, for example, to the time interval between two cardiac contractions, such as two ventricular contractions.

In the remainder of the description, detection signals caused by a malfunction of electronic hardware are also called detection signals characteristic of non-physiological activity.

In the remainder of the description, the term “electrode” or “probe” is used indifferently to refer to an electrode of an electrical device, such as for example a defibrillator.

Among the detection signals characteristic of non-physiological activity, there are and in a non-limiting way:
Signals generated by probe breakage;
Signals generated by a connector fault, for example at the probe connector in the housing;
Signals from a measurement noise;
Signals generated by an overdetection of a physiological signal in the vicinity of the measuring zone, said overdetection being caused by a hardware oversensitivity of the probe in the vicinity of the measuring zone;
Signals generated by an underdetection of a physiological signal in the vicinity of the measuring zone, said underdetection being caused by a hardware undersensitivity of the probe in the vicinity of the measuring zone,
Measurement interferences produced by a combination of signals between signals characteristic of physiological activity and signals not characteristic of physiological activity.

According to one embodiment, the first detection signal S_{D} is acquired by means of an electrode of an electrical device, such as for example an implantable cardioverter defibrillator.

In an example, in reference to FIG. 6, the first signal detected by the electrode along the time axis is the third detection signal S_{D-2} and the third signal detected by the electrode along the time axis is the first detection signal S_{D}.

According to one example, a plurality of detection signals S_{D}, S_{D-1}, S_{D-2} are acquired and each correspond to a characteristic cycle of cardiac electrical activity. For example, they are acquired successively in response to the detection of an electrical current by a probe implemented in the right ventricle of the heart of a patient. According to one case, the detection signals S_{D}, S_{D-1}, S_{D-2} correspond to three consecutive ventricular contractions. For example, the S_{D}, S_{D-1} and S_{D-2} signals correspond to signals characteristic of a right ventricular tachycardia. According to another example, the first detection signal S_{D} corresponds to a characteristic data of a non-physiological signal.

### Comparison Algorithm and Identification Information

In one embodiment, the method comprises implementing a comparison algorithm A_{COMP1} of the durations of the intervals of the acquired signals. In an example, in reference to FIG. 2, the comparison algorithm A_{COMP1} is implemented by a calculator K. For example, the calculator K is a calculator understood by a medical device such as an implantable cardioverter defibrillator (ICD).

Depending on the case, the comparison of these intervals with each other or with threshold values, makes it possible to determine whether the detection signals S_{D}, S_{D-1}, S_{D-2} correspond to physiological or physiopathological cardiac electrical activity or to a non-physiological signal.

For example, a succession of short cycles may correspond to a pathophysiological reality such as a ventricular fibrillation or ventricular tachycardia. According to another example, an alternation of long cycles and short cycles does not correspond to any physiological reality and the signals detected are associated with non-physiological information, for example caused by a hardware defect. It should be understood by “short cycle” a detection signal comprising an associated interval the value of which is less than a predefined threshold value, for example less than 200 ms. It should be understood by “long cycle” a detection signal comprising an associated interval the value of which is greater than a predefined threshold value, for example 550 ms.

Other limits or threshold values may be defined in a sequence of a long cycle followed by a short cycle, or a short cycle followed by a long cycle. Other examples are detailed hereinafter according to the interval comparison algorithms implemented.

According to the configuration of the algorithm, the threshold constraint may be implemented on the threshold of the long cycle (low threshold or high threshold with respect to an average value) or on the threshold of the short cycle (low or high threshold with respect to an average value). According to one example, the time intervals i, i−1, i−2 associated with the detection signals S_{D}, S_{D-1}, S_{D-2} are time intervals measured in milliseconds. However, any type of suitable time measurement unit is able to be used according to the cases of implementation of the invention.

### 1^{st} Comparison Algorithm

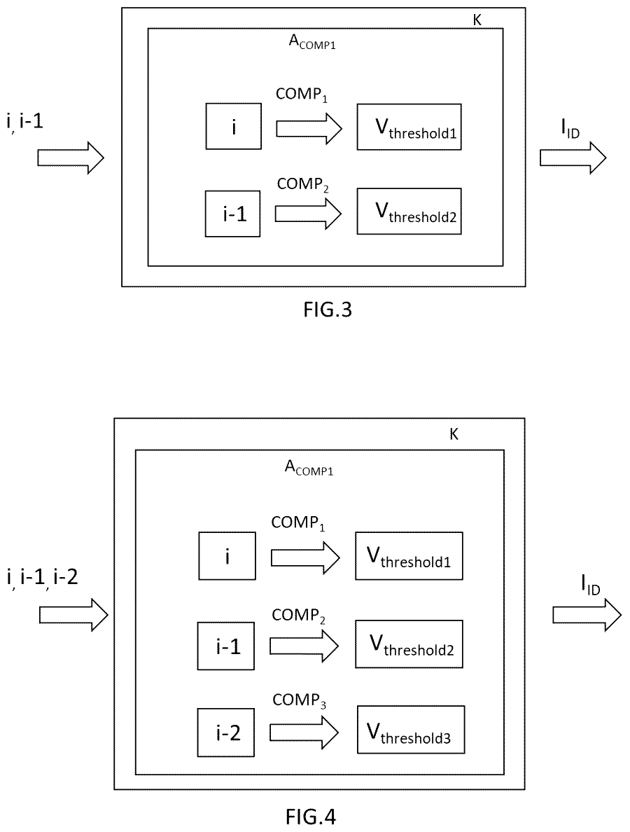

In one embodiment, the comparison algorithm A_{COMP1} comprises a plurality of steps. In reference to FIG. 3, the plurality of steps comprises, for example, implementing a first comparison COMP₁ of the first interval i with a first threshold V_{threshold1} and implementing a second comparison COMP₂ of the second interval i−1 with a second threshold V_{threshold2}. It should be understood by “comparison” of an interval with a threshold determining whether the numerical value associated with said interval is less than, greater than or equal to said threshold.

In an embodiment in reference to FIG. 4, the plurality of steps implemented by comparison algorithm A_{COMP1} comprises implementing a third comparison COMP₃ of the third interval i−2 with a third threshold V_{threshold3}. One advantage is to vary the first and second thresholds V_{threshold1}, V_{threshold2}, for example to detect noise over a larger interval, by adding an additional comparison criterion.

In an example, the thresholds V_{threshold1}, V_{threshold2} of the comparison algorithm A_{COMP1} when it comprises two comparison steps COMP1, COMP₂ are different from the thresholds of this same algorithm when it comprises three comparison steps COMP₁, COMP₂, COMP₃. For example, larger thresholds V_{threshold1}, V_{threshold2} may be taken in the implementation of the algorithm of comparison A_{COMP1} when the latter comprises three comparison steps. This embodiment is particularly advantageous in that it makes it possible to detect an anomaly on the detection signal S_{D} over a wider range of values, by implementing a third comparison step.

Thresholds V_{threshold1}, V_{threshold2} are, for example, determined following tests carried out on a plurality of data to determine optimized values. One advantage is to identify as many signals as possible that may be associated with noise and therefore potentially from a hardware defect, for example from a defective probe.

In one embodiment, the thresholds V_{threshold1} and V_{threshold2} are configurable by a user. Thresholds V_{threshold1}, V_{threshold2} are, for example, configurable via a user interface. For example, the user interface is accessible on a defibrillator configured to implement the method according to the invention. According to another case, the thresholds V_{threshold1}, V_{threshold2} are configurable from another device, such as a remote entity communicating with a medical device configured to implement the method according to the invention. In an example, the thresholds V_{threshold1}, V_{threshold2} are configured upstream of the integration of the comparison algorithm A_{COMP1} in a defibrillator configured to implement the method according to the invention.

According to one embodiment, the thresholds V_{threshold1}, V_{threshold2}, V_{threshold3}, V_{threshold4}, V_{threshold5}, V_{threshold6} and V_{threshold7} may each take any value in milliseconds, in particular in the list of following values:
[5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995]

According to one embodiment, thresholds V_{threshold1} and V_{threshold2} Of the first comparison algorithm, when it comprises only two thresholds, may take any combination of values in milliseconds from the following list of combinations of values:
[(5; 360), (10; 365), (15; 370), (20; 375), (25; 380), (30; 385), (35; 390), (40; 395), (45; 400), (50; 405), (55; 410), (60; 415), (65; 420), (70; 425), (75; 430), (80; 435), (85; 440), (90; 445), (95; 450), (100; 455), (105; 460), (110; 465), (115; 470), (120; 475), (125; 480), (130; 485), (135; 490), (140; 495), (145; 500), (150; 505), (155; 510), (160; 515), (165; 520), (170; 525), (175; 530), (180; 535), (185; 540), (190; 545), (195; 550), (200; 555), (205; 560), (210; 565), (215; 570), (220; 575), (225; 580), (230; 585), (235; 590), (240; 595), (245; 600), (255; 610), (265; 620), (275; 630), (285; 640), (295; 650), (305; 660), (315; 670), (325; 680), (335; 690), (345; 700), (355; 710), (365; 720), (375; 730), (385; 740), (395; 750), (405; 760), (415; 770), (425; 780), (435; 790), (445; 800), (455; 810), (465; 820), (475; 830), (485; 840), (495; 850), (505; 860), (515; 870), (525; 880), (535; 890), (545; 900), (555; 910), (565; 920), (575; 930), (585; 940), (595; 950), (605; 960), (615; 970), (625; 980), (635; 990)]

According to one embodiment, the thresholds implemented in one of the comparison algorithms A_{COMP1}, A_{COM2} when they comprise 3 thresholds, such as thresholds V_{threshold1}, V_{threshold2} and V_{threshold3} or thresholds V_{threshold4}, V_{threshold5} and V_{threshold6} may take any of the values from the list of combinations of the following values:
(40; 250; 250), (45; 255; 255), (50; 260; 260), (55; 265; 265), (60; 270; 270), (65; 275; 275), (70; 280; 280), (75; 285; 285), (80; 290; 290), (85; 295; 295), (90; 300; 300), (95; 305; 305), (100; 310; 310), (105; 315; 315), (110; 320; 320), (115; 325; 325), (120; 330; 330), (125; 335; 335), (130; 340; 340), (135; 345; 345), (140; 350; 350), (145; 355; 355), (150; 360; 360), (155; 365; 365), (160; 370; 370), (165; 375; 375), (170; 380; 380), (175; 385; 385), (180; 390; 390), (185; 395; 395), (190; 400; 400), (195; 405; 405), (200; 410; 410), (205; 415; 415), (210; 420; 420), (215; 425; 425), (220; 430; 430), (225; 435; 435), (230; 440; 440), (235; 445; 445), (240; 450; 450), (245; 455; 455), (250; 460; 460), (255; 465; 465), (260; 470; 470), (265; 475; 475), (270; 480; 480), (275; 485; 485), (280; 490; 490), (285; 495; 495), (290; 500; 500), (300; 510; 510), (310; 520; 520), (320; 530; 530), (330; 540; 540), (340; 550; 550), (350; 560; 560), (360; 570; 570), (370; 580; 580), (380; 590; 590), (390; 600; 600), (400; 610; 610), (410; 620; 620), (420; 630; 630), (430; 640; 640), (440; 650; 650), (450; 660; 660), (460; 670; 670), (470; 680; 680), (480; 690; 690), (490; 700; 700), (500; 710; 710), (510; 720; 720), (520; 730; 730), (530; 740; 740), (540; 750; 750), (550; 760; 760), (560; 770; 770), (570; 780; 780), (580; 790; 790), (590; 800; 800), (600; 810; 810), (610; 820; 820), (620; 830; 830), (630; 840; 840), (640; 850; 850), (650; 860; 860), (660; 870; 870), (670; 880; 880), (680; 890; 890), (690; 900; 900),

In this version of the algorithm, the thresholds are raised and less constrained, which makes it possible to obtain information that would not have been detected in the implementation with the first version of the algorithm comprising two more constrained thresholds (lower and/or higher limits).

However, the above examples are in no way limiting and the thresholds may be configured with different values.

According to different embodiments, the values of the different thresholds V_{threshold1}, V_{threshold2}, V_{threshold3}, V_{threshold4}, V_{threshold5}, V_{threshold6} and V_{threshold7} are within value ranges between 1 millisecond and 1 second.

According to an example wherein the first comparison algorithm A_{COMP1} comprises only thresholds V_{threshold1} and V_{threshold2}, the thresholds V_{threshold1} and V_{threshold2} are for example between 100 milliseconds and 700 milliseconds.

According to another example, the threshold V_{threshold1} is between 100 milliseconds and 300 milliseconds and the threshold V_{threshold2} is between 400 milliseconds and 600 milliseconds when the first comparison algorithm A_{COMP1} comprises two comparison steps.

According to another example, the threshold V_{threshold1} is between 200 milliseconds and 300 milliseconds and the thresholds V_{threshold2} and V_{threshold3} are between 400 milliseconds and 450 milliseconds when the first comparison algorithm A_{COMP1} comprises three comparison steps.

According to another example, the threshold V_{threshold1} is between 200 milliseconds and 250 milliseconds and the thresholds V_{threshold2} and V_{threshold3} are for example between 400 milliseconds and 500 milliseconds when the first comparison algorithm A_{COMP1} comprises three comparison steps.

According to another example, the thresholds V_{threshold4}, V_{threshold5} and V_{threshold6} of the second comparison algorithm A_{COMP2} are between 200 milliseconds and 600 milliseconds.

In another example, threshold V_{threshold4} is configured with a value between 200 milliseconds and 300 milliseconds and thresholds V_{threshold5} and V_{threshold6} are configured with values between 400 milliseconds and 500 milliseconds.

In another example, threshold V_{threshold4} is configured with a value between 200 milliseconds and 250 milliseconds and thresholds V_{threshold5} and V_{threshold6} are configured with values between 400 milliseconds and 450 milliseconds.

According to one embodiment, the predefined thresholds are replaced with dynamic thresholds defined according to prior time intervals. An example is the verification and detection of a long cycle followed by a short cycle relative to the length of the interval of the long cycle. For example, the method would make it possible to identify a time interval of the short cycle that is 20% shorter than the interval of the long cycle.

According to different cases, the values chosen for the thresholds in the comparison algorithms A_{COMP1}, A_{COMP2} depend on the nature of the patient. It should be understood by “nature” of the patient that the thresholds chosen will for example be different according to age, weight, gender, pathologies (cardiac or not), or any other characteristic of the patient. Thus, the values chosen for the thresholds depend on various factors inherently related to the patient on a case-by-case basis, so as to advantageously obtain a more suitable detection of an item of identification information I_{id} characteristic of a non-physiological signal for a given patient.

Combination of the 1^{st} and of the 2^{nd} Comparison Algorithms

In one embodiment, the method comprises implementing a second comparison algorithm A_{COMP2}. The second comparison algorithm A_{COMP2} comprises a plurality of comparison steps. According to one example, the second comparison algorithm A_{COMP2} is implemented consecutively to the implementation of the first comparison algorithm A_{COMP1}. In another example, the two comparison algorithms A_{COMP1}, A_{COMP2} are implemented in parallel or jointly. According to another case, the two comparison algorithms A_{COMP1}, A_{COMP2} are implemented independently of each other.

Recall that the first algorithm may comprise two or three thresholds. The second algorithm comprises at least three thresholds. When the two algorithms are implemented jointly on the same measurements, they are then preferably configured so that the first algorithm comprises two thresholds and the second algorithm comprises three thresholds.

One advantage of this configuration is to obtain different criteria for classifying the time anomalies of the signals detected (overdetection, arrhythmia, measurement artifact, etc.). Thus, if an algorithm detects a false positive or misses a detection, the implementation of the second algorithm increases the probability of detections of anomalies and their classification. Thus, an algorithm favors the definition of constrained time limits (high or low values), and another algorithm favors the persistence of an anomaly over several intervals.

In an example shown in FIG. 5, the first comparison algorithm A_{COMP1} comprises the first comparison COMP₁ of the first interval i with the first threshold V_{threshold1} and the second comparison COMP₂ of the second interval i−1 with the second threshold V_{threshold2}. In this example, the second comparison algorithm A_{COMP2} comprises a fourth comparison of the first interval i with a fourth threshold V_{threshold4}, a fifth comparison of the second interval i−1 with a fifth threshold V_{threshold5} and a sixth comparison of the third interval i−2 with a sixth threshold V_{threshold6}. In this example, the third comparison COMP₃ is not implemented and therefore the first algorithm A_{COMP1} only comprises two comparison steps.

In one embodiment, the values of thresholds V_{threshold1}, V_{threshold2}, V_{threshold3}, V_{threshold4}, V_{threshold5}, V_{threshold6} and V_{threshold7} are interchangeable with each other.

In one embodiment, the comparison algorithms A_{COMP1}, A_{COMP2} are configurable. In this case, for example, one or more comparison operators implemented by said algorithms A_{COMP1}, A_{COMP2} are able to be modified.

### Thresholds Defined by an Average or a Median

In an embodiment, at least one threshold among the thresholds V_{threshold1}, V_{threshold2}, V_{threshold3}, V_{threshold4}, V_{threshold5}, V_{threshold6} and V_{threshold7} comprises a weighted average. For example, the weighted average comprises a weighted average of the intervals i, i−1, i−2 and i−3 cycle to cycle. One advantage is to identify particular events, such as a frequency jump or instability of the system.

In an embodiment, at least one threshold among thresholds V_{threshold1}, V_{threshold2}, V_{threshold3}, V_{threshold4}, V_{threshold5}, V_{threshold6} and V_{threshold7} comprises a median value. For example, the median value comprises a median value of the intervals i, i−1, i−2 and i−3.

### Thresholds Defined by Functions

In an embodiment, at least one threshold among thresholds V_{threshold1}, V_{threshold2}, V_{threshold3}, V_{threshold4}, V_{threshold5}, V_{threshold6} and V_{threshold7} comprises a mathematical function f. According to one case, the mathematical function f is a function that depends on a time interval i, i−1, i−2 or i−3, or a mathematical function that depends on several of these time intervals. In other cases, the mathematical function f depends on variables other than the time intervals i, i−1, i−2 or i−3. The mathematical function f may also comprise a combination of several mathematical functions together.

In another example, the mathematical function f comprises a linear combination of the time intervals i, i−1 and i−2 such as f=a(i)+b(i−1)+c(i−2) where a, b and c are constants. In this case, for example, the interval i associated with the detection signal S_{D} is compared with the value taken by the function f according to the values of the time intervals i, i−1 and i−2 to determine whether the value of the time interval i is greater or less than the value taken by said function f. The item of identification information I_{id} is then assigned to the detection signal S_{D} according to the result of the comparison of the interval i with said mathematical function f.

In another example, the mathematical function f comprises a linear combination of time intervals i, i−1, i−2 and i−3 such that f=a(i)+b(i−1)+c(i−2)+d (i−3) where a, b, c and d are constants.

According to one embodiment, several mathematical functions f are assigned to different thresholds among the thresholds V_{threshold1}, V_{threshold2}, V_{threshold3}, V_{threshold4}, V_{threshold5}, V_{threshold6} and V_{threshold7}. Thus, the time intervals i, i−1, i−2 and i−3 are compared with different mathematical functions f to assign the item of identification information I_{id} to the detection signal S_{D}.

According to one embodiment, at least one of the comparison algorithms A_{COMP1}, A_{COMP2} comprises constant thresholds and variable thresholds. “Constant” threshold means the assigning of a constant value to one of the thresholds V_{threshold1}, V_{threshold2}, V_{threshold3}, V_{threshold4}, V_{threshold5}, V_{threshold6} Or V_{threshold7}. “Variable” threshold means the assigning of a mathematical function f to one of the thresholds V_{threshold1}, V_{threshold2}, V_{threshold3}, V_{threshold4}, V_{threshold5}, V_{threshold6} Or V_{threshold7}.

In an example, the first comparison algorithm A_{COMP1} comprises the first threshold V_{threshold1} to which a mathematical function f(i, i−1) is assigned and the second threshold V_{threshold2} to which a constant value is assigned, for example 500 ms. Thus, the first time interval i is compared with the mathematical function f which depends on the first and second time intervals i, i−1 and the second time interval is compared with a constant value. The item of identification information I_{id} is then assigned to the detection signal S_{D} according to the result of these comparisons.

According to one case, in reference to FIG. 8, at least one of the two comparison algorithms A_{COMP1}, A_{COMP2} comprises a triggering zone Z_{C}. The type of item of identification information I_{id} allocated to the detection signal S_{D} depends on the fact that the result of the different comparisons implemented by one of the algorithms A_{COMP1}, A_{COMP2} corroborate with the triggering zone Z_{C} of one of the algorithms.

For example, in the case where the thresholds V_{threshold1} and V_{threshold2} of the first comparison algorithm A_{COMP1} are constants, the triggering zone Z_{C} corresponds, for example, to the zone wherein the first time interval i is less than the first threshold V_{threshold1} and the second time interval i−1 is greater than the second threshold V_{threshold2}.

Thus, the triggering zone Z_{C} represented by a graphic zone in FIG. 8 comprises all the points corresponding to the cases for which the criteria of one of the comparison algorithms A_{COMP1}, A_{COMP2} are verified and for which an item of specific identification information I_{id} is assigned to the detection signal S_{D}, for example an item of anomaly information characteristic of a non-physiological signal.

The method comprises assigning an item of identification information I_{id} to the first detection signal S_{D}. The item of identification information I_{id} comprises, for example, information on the nature of the detection signal S_{D}. It should be understood by “nature of the detection signal S_{D}” the fact that a detection signal S_{D} corresponds to a physiological or physiopathological reality or that the detection signal S_{D} has no physiological or physiopathological reality and is, for example, noise.

In one embodiment, the item of identification information I_{id} comprises physiological information. According to one example, the physiological information comprises information on a natural beat rate of the heart.

In one embodiment, the item of identification information I_{id} comprises pathophysiological information. According to various examples, the pathophysiological information comprises information on a cardiac arrhythmia such as bradycardia, tachycardia or even fibrillation. This information corresponds, for example, to an arrhythmia detected by a probe positioned in a heart chamber of a patient, such as the right ventricle.

However, the type of item of identification information I_{id} associated with the detection signal S_{D} is not limited to the above examples and may comprise any type of item of information associated with a particular heart rate identified and associated with the first detection signal S_{D}.

In one embodiment, the item of identification information I_{id} comprises an item of anomaly information. According to one case, the item of anomaly information characterizes a non-physiological signal, for example from the malfunction of an electrode. In one example, the item of anomaly information is associated with the first detection signal S_{D} when said first detection signal S_{D} is generated by a device comprising a defective electrode and manifests in the form of noise on the detection channel.

### Overdetection Phenomenon and Third Criterion

In one embodiment, the item of anomaly information comprises an item of “overdetection” information. It should be understood by “overdetection” that the detection of a signal having an origin different from the S_{D} signal due to the contraction of a chamber wherein the probe is implemented, and at the same time different from a signal characteristic of non-physiological information, such as a signal due to a break in the probe. According to various examples, the item of “overdetection” information comprises information on the detection of cardiac electrical activity resulting from ventricular relaxation, or even from contraction of the atrium, when the probe is positioned in the right ventricle. More generally, the item of “overdetection” information is assigned to the first detection signal S_{D} when the latter does not result from a contraction of the chamber wherein the probe is implemented and the first detection signal S_{D} is not characteristic of a non-physiological signal, for example resulting from a hardware defect.

In a variant, the item of anomaly information comprises an item of “underdetection” information. It should be understood by “underdetection” that part of the cardiac electrical activity has not been correctly detected by the probe.

According to one embodiment, the comparison algorithm A_{COMP2} comprises the implementation of a seventh comparison step COMP₇.

In one embodiment, the comparison algorithm A_{COMP1} comprises implementing the seventh comparison step COMP₇.

For example, the seventh comparison step COMP₇ comprises comparing the fourth time interval i−3 with a seventh threshold V_{threshold7}. In this case, the second algorithm A_{COMP2} then comprises four comparison thresholds. When the first comparison algorithm comprises the implementation of the seventh comparison step COMP₇, it then comprises three or four comparison thresholds. This implementation is particularly advantageous in distinguishing characteristic signals of probe breakage problems from characteristic signals from overdetection phenomena. Indeed, overdetection phenomena not related to probe breakages are mostly characterized by a single “short” cycle, i.e. less than a predefined time interval. Therefore, to ensure that the phenomenon detected is indeed due to a break in a probe, it must be ensured that several short cycles occur in succession. In other words, it is sought to ensure that an interval following a given interval below a predefined threshold is also below a predefined threshold (for example the same threshold).

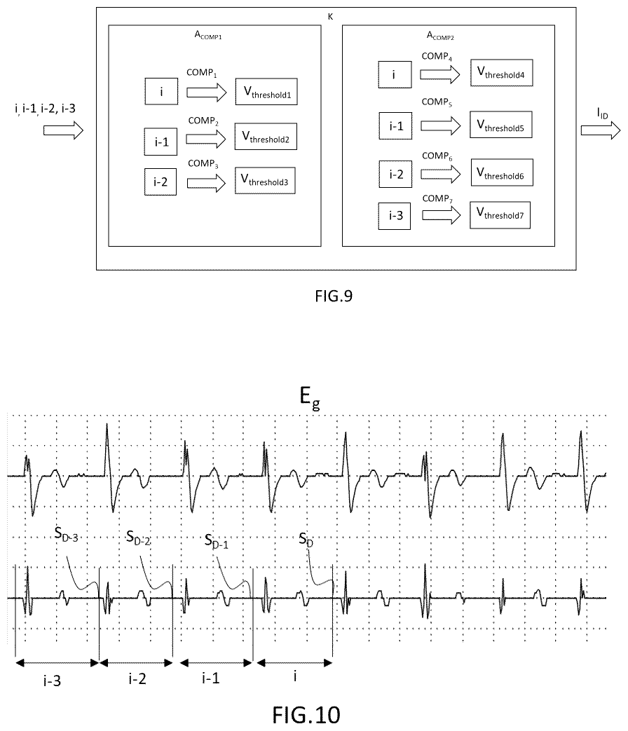

To illustrate this mode, take the example shown in FIG. 9 where the first comparison algorithm A_{COMP1} comprises three comparison steps COMP₁, COMP₂ and COMP₃ and the second comparison algorithm A_{COMP2} comprises four comparison steps COMP₄, COMP₅, COMP₆ and COMP₇. In this case, the COMP₁, COMP₂ comparisons for the first algorithm A_{COMP1} and the comparisons COMP₄, COMP₅ for the second algorithm are implemented to ensure that the successive time intervals i and i−1 are each below predefined thresholds (and therefore characteristics of short cycles). Therefore, the implementation of the algorithm makes it possible to both optimize noise detection by implementing two algorithms successively taking into account several comparison criteria, and also to ensure that the noise detected is indeed the result of a break in the probe and not an overdetection phenomenon.

According to another embodiment, the first comparison algorithm A_{COMP1} comprises only two comparison steps COMP₁, COMP₂ and the second comparison algorithm A_{COMP2} comprises four comparison steps COMP₄, COMP₅, COMP₆, COMP₇. In this case, the distinction between the overdetection phenomenon and the probe breakage problem is studied only via the second comparison algorithm A_{COMP2}. In this example, it is verified that two successive short cycles follow two successive long cycles to determine a probe breakage problem.

According to another embodiment, the first comparison algorithm A_{COMP1} comprises three comparison steps COMP₁, COMP₂, COMP₃ and the second comparison algorithm A_{COMP2} comprises three comparison steps COMP₄, COMP₅, COMP₆. In this case, the distinction between overdetection and probe breakage is for example only studied through an algorithm among the first and second comparison algorithms A_{COMP1}, A_{COMP2}. In this case, for example, it is verified that a long cycle is followed by two successive short cycles.

According to one embodiment, the first comparison algorithm A_{COMP1} comprises two comparison steps COMP₁, COMP₂ and the second comparison algorithm A_{COMP2} comprises three comparison steps COMP₄, COMP₅, COMP₆. In this case, the distinction between the phenomenon of overdetection and the problem of probe breakage is for example only studied via the second comparison algorithm A_{COMP2}.

According to one embodiment, the thresholds implemented in the comparison algorithm A_{COMP1} or the comparison algorithm A_{COMP2} when they comprise 4 thresholds, such as the thresholds V_{threshold4}, V_{threshold5}, V_{threshold6} and V_{threshold7} may take any value from the list of combinations of the following values:
(40; 40; 250; 250), (45; 45; 255; 255), (50; 50; 260; 260), (55; 55; 265; 265), (60; 60; 270; 270), (65; 65; 275; 275), (70; 70; 280; 280), (75; 75; 285; 285), (80; 80; 290; 290), (85; 85; 295; 295), (90; 90; 300; 300), (95; 95; 305; 305), (100; 100; 310; 310), (105; 105; 315; 315), (110; 110; 320; 320), (115; 115; 325; 325), (120; 120; 330; 330), (125; 125; 335; 335), (130; 130; 340; 340), (135; 135; 345; 345), (140; 140; 350; 350), (145; 145; 355; 355), (150; 150; 360; 360), (155; 155; 365; 365), (160; 160; 370; 370), (165; 165; 375; 375), (170; 170; 380; 380), (175; 175; 385; 385), (180; 180; 390; 390), (185; 185; 395; 395), (190; 190; 400; 400), (195; 195; 405; 405), (200; 200; 410; 410), (205; 205; 415; 415), (210; 210; 420; 420), (215; 215; 425; 425), (220; 220; 430; 430), (225; 225; 435; 435), (230; 230; 440; 440), (235; 235; 445; 445), (240; 240; 450; 450), (245; 245; 455; 455), (250; 250; 460; 460), (255; 255; 465; 465), (260; 260; 470; 470), (265; 265; 475; 475), (270; 270; 480; 480), (275; 275; 485; 485), (280; 280; 490; 490), (285; 285; 495; 495), (320; 320; 530; 530), (300; 300; 510; 510), (290; 290; 500; 500), (330; 330; 540; 540), (310; 310; 520; 520), (340; 340; 550; 550), (350; 350; 560; 560), (360; 360; 570; 570), (370; 370; 580; 580), (380; 380; 590; 590), (390; 390; 600; 600), (400; 400; 610; 610), (410; 410; 620; 620), (420; 420; 630; 630), (430; 430; 640; 640), (440; 440; 650; 650), (450; 450; 660; 660), (460; 460; 670; 670), (470; 470; 680; 680), (480; 480; 690; 690), (490; 490; 700; 700), (500; 500; 710; 710), (510; 510; 720; 720), (520; 520; 730; 730), (530; 530; 740; 740), (540; 540; 750; 750), (550; 550; 760; 760), (560; 560; 770; 770), (570; 570; 780; 780), (580; 580; 790; 790), (590; 590; 800; 800), (600; 600; 810; 810), (610; 610; 820; 820), (620; 620; 830; 830), (630; 630; 840; 840), (640; 640; 850; 850), (650; 650; 860; 860), (660; 660; 870; 870), (670; 670; 880; 880), (680; 680; 890; 890), (690; 690; 900; 900).

In this version of the algorithm, the introduction of an additional threshold advantageously makes it possible to distinguish the signals due to a phenomenon of overdetection from signals due to a probe breakage problem. For example, a succession of two short cycles preceded by a succession of two long cycles may correspond to a probe break.

According to one embodiment, the thresholds implemented in one of the comparison algorithms A_{COMP1}, A_{COM2} when they comprise 3 thresholds, such as thresholds V_{threshold1}, V_{threshold2} and V_{threshold3} or thresholds V_{threshold4}, V_{threshold5} and V_{threshold6} may take any of the values from the list of combinations of the following values:
(40; 40; 250), (45; 45; 255), (50; 50; 260), (55; 55; 265), (60; 60; 270), (65; 65; 275), (70; 70; 280), (75; 75; 285), (80; 80; 290), (85; 85; 295), (90; 90; 300), (95; 95; 305), (100; 100; 310), (105; 105; 315), (110; 110; 320), (115; 115; 325), (120; 120; 330), (125; 125; 335), (130; 130; 340), (135; 135; 345), (140; 140; 350), (145; 145; 355), (150; 150; 360), (155; 155; 365), (160; 160; 370), (165; 165; 375), (170; 170; 380), (175; 175; 385), (180; 180; 390), (185; 185; 395), (190; 190; 400), (195; 195; 405), (200; 200; 410), (205; 205; 415), (210; 210; 420), (215; 215; 425), (220; 220; 430), (225; 225; 435), (230; 230; 440), (235; 235; 445), (240; 240; 450), (245; 245; 455), (250; 250; 460), (255; 255; 465), (260; 260; 470), (265; 265; 475), (270; 270; 480), (275; 275; 485), (280; 280; 490), (285; 285; 495), (290; 290; 500), (300; 300; 510), (310; 310; 520), (320; 320; 530), (330; 330; 540), (340; 340; 550), (350; 350; 560), (360; 360; 570), (370; 370; 580), (380; 380; 590), (390; 390; 600), (400; 400; 610), (410; 410; 620), (420; 420; 630), (430; 430; 640), (440; 440; 650), (450; 450; 660), (460; 460; 670), (470; 470; 680), (480; 480; 690), (490; 490; 700), (500; 500; 710), (510; 510; 720), (520; 520; 730), (530; 530; 740), (540; 540; 750), (550; 550; 760), (560; 560; 770), (570; 570; 780), (580; 580; 790), (590; 590; 800), (600; 600; 810), (610; 610; 820), (620; 620; 830), (630; 630; 840), (640; 640; 850), (650; 650; 860), (660; 660; 870), (670; 670; 880), (680; 680; 890), (690; 690; 900),
One advantage of using the above-mentioned threshold values in either of the comparison algorithms A_{COMP1}, A_{COMP2} is to maintain good probe breakage detection sensitivity while improving the distinction between overdetection phenomena and probe breakage problems.

In the event that the sensitivity of probe breakages is affected by the implementation of a new criterion, this sensitivity will tend to improve over time as more noise will be detected; and therefore the probability of detection of succession of short intervals will increase.

In one embodiment, the type of item of identification information I_{id} associated with the detection signal S_{D} depends on the result of one or more comparisons performed by the comparison algorithm A_{COMP1}.

According to one example, the first interval i is less than the first threshold V_{threshold1} and the second interval i−1 is less than the second threshold V_{threshold2}. For example, the first comparison algorithm A_{COMP1} has been configured such that, if the criteria for comparing the intervals i, i−1 with said thresholds V_{threshold1}, V_{threshold2} are not met, i.e. the first interval i is greater than the threshold value V_{threshold1} or the second interval i−1 is less than the second threshold value V_{threshold2}, the first detection signal S_{D} cannot have a physiological or pathophysiological reality. Therefore, an item of identification information I_{id} comprising an item of anomaly information is assigned to the first detection signal S_{D}.

According to another illustrative example, the first interval i is less than the first threshold V_{threshold1} and the second interval i−1 is greater than the second threshold V_{threshold2}. For example, the first comparison algorithm A_{COMP1} has been configured such that, if the criteria for comparing the intervals i, i−1 with said thresholds V_{threshold1}, V_{threshold2} are met, i.e. the first interval i is less than the threshold value V_{threshold1} or the second interval i−1 is greater than the second threshold value V_{threshold2}, the first detection signal S_{D} cannot have a physiological or physiopathological reality. Therefore, an item of identification information I_{id} comprising an item of anomaly information is assigned to the first detection signal S_{D}.

In one embodiment, the type of item of identification information I_{id} assigned to the first detection signal S_{D} depends on the result of the comparisons implemented by at least one of the two comparison algorithms A_{COMP1}, A_{COMP2}. Advantageously, the fact that the conditions of only one of the two comparison algorithms A_{COMP1}, A_{COMP2} are not met results in the assigning of an item of anomaly information to the first detection signal S_{D}. Another advantage is to make noise detection on the detection channel more efficient; by implementing a higher number of comparison criteria.

In one embodiment, the item of identification information I_{id} is stored in a memory space. The memory space comprises, for example, a storage space. For example, the memory space is implemented on a medical device, such as an implantable cardioverter defibrillator.

In one embodiment, the method comprises generating a notification or an A₁ alert when the item of identification information I_{id} comprises an item of anomaly information. Depending on the case, the alert A₁ is stored in the memory space.

In an embodiment, the notification or alert A₁ is generated following the assigning of several items of identification information I_{id} each comprising an item of anomaly information. According to one example, the alert or notification A₁ is transmitted when five items of identification information I_{id} comprise an item of anomaly information. One advantage is to limit the emission of the alert to the repeated detection of non-physiological events. This makes it possible to avoid, for example, the “false positives” of non-physiological events and thus to avoid an excessively frequent or inadequate alert escalation.

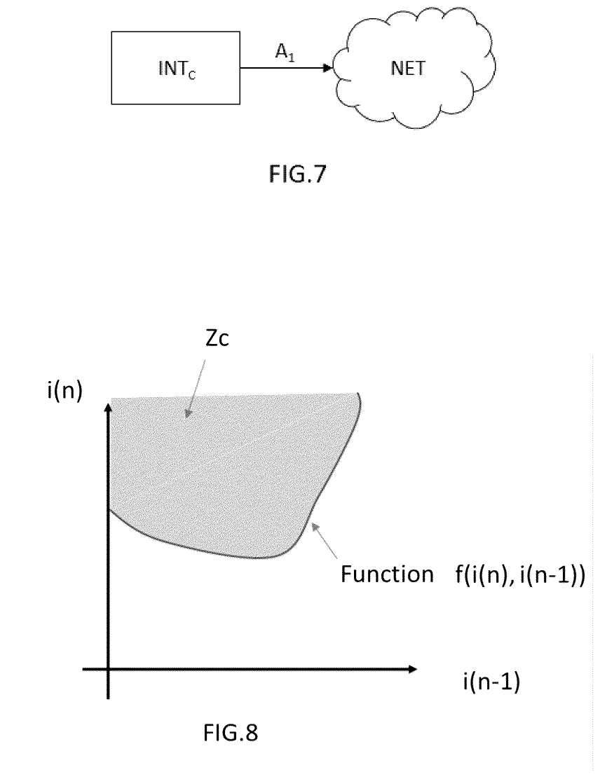

In one embodiment, in reference to FIG. 7, alert A₁ is transmitted to equipment of a data network NET. For example, the alert A₁ is transmitted by means of a signal emitted by a communication interface INT_{C}. The equipment of a data network NET comprises for example a remote server. In an example, alert A₁ is transmitted via a telemedicine alert. Advantageously, the alert A₁ thus transmitted may then be read by a user, for example medical personnel, to observe the malfunction of the device of a patient and act accordingly. The risks of patient death due to electrode malfunction are advantageously reduced.

### Cascade Thresholds for Algorithms

In one embodiment, at least one of the two comparison algorithms A_{COMP1}, A_{COMP2} comprises other comparison steps than steps COMP₁, COMP₂, COMP₃, COMP₄, COMP₅, COMP₆, COMP₇.

Thus, the implementation of one or two algorithms comprising time interval comparison steps associated with detection signals falls within the scope of the invention; regardless of the number of comparison steps implemented by the algorithms.

### Continuous Cycle-to-Cycle Analysis

In one embodiment, the method comprises acquiring a plurality of detection signals S_{D} continuously. Recall that a time interval is associated with each new detection signal S_{D} acquired. This time interval therefore corresponds to the time interval separating it from a prior signal, for example a signal S_{D-1} preceding it.

In this continuous analysis process, the method according to the invention is performed continuously by considering successive intervals by “sliding” the compared intervals throughout the analysis.

Thus, for each acquired detection signal S_{D}, a comparison of the time interval i that is associated with it with time intervals associated with prior signals, for example by means of the comparison algorithm A_{COMP1}, makes it possible to obtain the item of identification information lip of said detection signal Sp. Therefore, the continuous comparison of the intervals associated with each new detection signal acquired and the intervals associated with prior signals, for example with thresholds using one of the comparison algorithms A_{COMP1}, A_{COMP2}, makes it possible to obtain the item of identification information I_{ID} for each new detection signal S_{D} acquired.

This mode is particularly advantageous to determine a noise incidence rate on the detection channel, for example by observing a frequency of appearance of an item of anomaly information associated with each item of identification information I_{id}. Thus, this cycle-to-cycle analysis makes it possible to extract spectral information linked on the one hand to the rate of appearance of the item of identification information produced by the method of the invention and on the other hand to the rate of appearance of arrhythmia anomalies caused by physiological signals. This spectral analysis allows corroborating information and hence, for example, confirming a hardware fault.

### Electrogram

In one embodiment, the method according to the invention comprises generating an electrogram E_{G}. The electrogram E_{G} comprises for example a recording of the cardiac activity of a patient over a given time interval.

According to one embodiment, in reference to FIG. 6, the electrogram E_{G} comprises a graphical representation of the three detection signals S_{D}, S_{D1}, S_{D2} and the time intervals i, i−1 and i−2 associated with said detection signals.

According to another embodiment shown in FIG. 10, the electrogram E_{G} comprises a graphical representation of four detection signals S_{D}, S_{D1}, S_{D2}, S_{D3} and time intervals i, i−1, i−2, i−3 associated with said detection signals.

In one embodiment, the method comprises acquiring an image of the electrogram E_{G}. According to one case, the acquisition of the image of the electrogram E_{G} takes place when the item of identification information I_{id} comprises an item of anomaly information. In another case, the acquisition of an image of the electrogram E_{G} takes place periodically, for example according to a predefined period. In an example, the image of the electrogram E_{G} is stored in a memory space.

According to one embodiment, the image of the electrogram E_{G} is transmitted to equipment of a data network NET. For example, the image of the electrogram E_{G} is transmitted via the communication interface INT_{C}. For example, the image of the electrogram E_{G} is automatically transmitted following its recording in the memory space.

In one embodiment, the image of the electrogram E_{G} is transmitted at the same time as alert A₁. The image of the electrogram E_{G} is for example via a telemedicine alert. One advantage is to allow skilled personnel to familiarize themselves with the graphical representation of the signals in order to be able, a posteriori, to confirm or invalidate the presence of noise on the detection channel of the probe.

In one embodiment, transmitting the image of the electrogram E_{G} to an equipment of a data network NET automatically causes it to be deleted from the memory space on which said image is stored. One advantage is to free up memory space when the image of the electrogram E_{G} has been transmitted to the competent medical personnel for analysis. In another case, the recorded images are periodically deleted from the memory space, according to a predefined time period.

In one embodiment, the method comprises deactivating at least one comparison algorithm among algorithms A_{COMP1}, A_{COMP2}. In an example, the deactivating of the algorithm is effective during a first time period. For example, deactivation of the comparison algorithm A_{COMP1} is implemented following reception of a deactivation command. According to one case, the deactivation command received was emitted beforehand by a remote entity.

In an embodiment, the alert A₁ and the electrogram E_{G} are transmitted via a telemedicine alert to a remote entity such as equipment of a data network NET. For example, the signals of the electrogram E_{G} are analyzed by medical personnel to determine whether the detection signal S_{D} corresponds to the detection of a signal characteristic of non-physiological electrical activity. A command to deactivate the comparison algorithm is then received if the alert A₁ emitted is due to a false positive. One advantage is to avoid excessively frequent escalation of alerts due to false positives.

### Electrical Device (Pacemaker and Defibrillator)

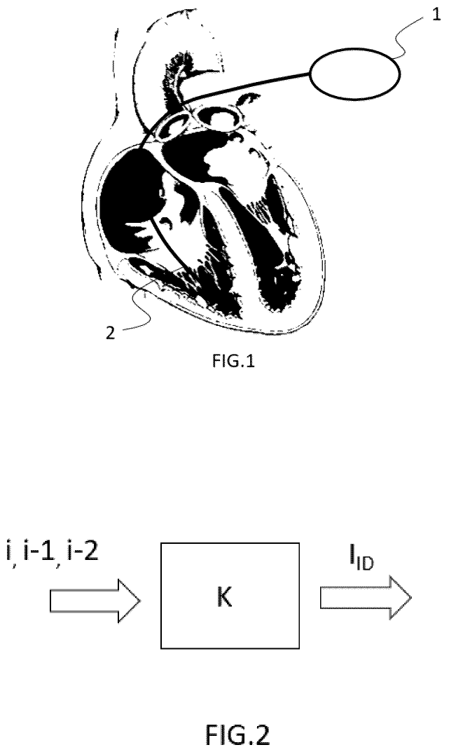

According to one embodiment, in reference to FIG. 1, the first detection signal S_{D} comprises a signal detected by an electrode 2 of an electrical device 1. For example, the electrical device 1 comprises a medical device such as an implantable cardioverter defibrillator or a “pacemaker”. According to another example, the electrical device 1 comprises a subcutaneous device.

For example, the electrical device 1 is implemented in the body of a patient. In one example, the electrical device 1 is intended to stimulate the heart muscles. For example, stimulation is performed when the heart rhythm of the patient is considered characteristic of arrhythmia, e.g. in case of ventricular tachycardia or ventricular fibrillation, or in case of ventricular bradycardia.

According to several examples, the electrical device 1 comprises a single-chamber, two-chamber, or three-chamber implantable cardioverter defibrillator. The number of chambers indicates the number of probes that connect the electrical device 1 to heart chambers, for example to the ventricles and atria. One advantage is to stimulate a given number of heart chambers.

More generally, any type of device, implantable or not, intended to measure or stimulate the cardiac activity of a patient is able to be used for the implementation of the method according to the invention.

The electrical device 1 requires a power supply to operate. According to one example, the electrical power supply implemented to power the electrical device 1 comprises a battery. For example, the battery comprises lithium-ion technology. However, the above example is in no way limiting and any type of battery technology is able to be used to power the electrical device 1.

According to several examples, the electrical device 1 comprises various components. According to one case, the electrical device 1 comprises a housing. For example, the housing comprises a battery, sensors, electronic circuits or a memory for recording data.

In one embodiment, the electrical device 1 comprises an electrical energy storage element. The electrical device 1 comprises for example a capacitor. According to one example, a defined amount of electrical energy is stored in the electrical energy storage element. For example, the amount of stored electrical energy is discharged to automatically deliver a therapeutic shock to a patient in response to detection of cardiac arrhythmia.

In one embodiment, the electrical device 1 comprises a communication interface INT_{C}. According to one case, the communication interface INT_{C} is configured to exchange data with remote equipment, such as equipment of a data network NET. The communication interface INT_{C} comprises for example a remote transmitter. According to one case, the communication interface INT_{C} is configured to exchange data automatically and at regular intervals with remote equipment.

According to an embodiment, the communication interface INT_{C} is configured to exchange data with a transmitter/receiver device. In an example, the transmitter/receiver device is configured to receive data transmitted by the communication interface INT_{C} and to automatically transmit the data received to remote equipment. One advantage is to be able to send data acquired by the device to remote equipment, for example to a remote control center.

### Probe(s)

According to one embodiment, in reference to FIG. 1, the electrical device 1 comprises a probe 2. In one example, the probe 2 makes it possible to connect the electrical device 1 to a heart chamber of a patient.

In one case, the probe 2 comprises electronic components. The electronic components are, for example, able to deliver electrical pulses calibrated in frequency and amplitude.

According to several examples, the probe 2 consists of various materials and/or alloys. For example, the alloys comprise titanium and/or carbon. However, these examples are not limiting and the probe 2 is able to comprise any type of material or alloys.

According to other examples, other probes are able to be used to measure cardiac electrical activity, such as an electrode positioned in the left ventricle of the patient. More generally, any type of probe connected to the heart muscle or any type of extra-cardiac probe, for example connected to a subcutaneous defibrillator, is able to be implemented in the scope of the invention.

According to a preferred embodiment, the electrical device 1 comprises a single probe 2. For example, the probe 2 is positioned in the right ventricle of the heart of the patient. This case is particularly advantageous in that an abnormality of the right ventricular electrode is able to have dramatic consequences up to and including death of the patient. In addition, given that at present, the majority of patients are equipped with a probe positioned in the right ventricle without necessarily being equipped with another additional probe, the invention has particular interest in being implemented from the signals measured by a single probe positioned in the right ventricle of a patient.

According to another example, the probe is a subcutaneous electrode.

In one embodiment, the electrical device 1 comprises a plurality of probes. According to one example, the pacemaker 1 comprises two probes each implemented in a ventricle of the heart of the patient. According to another example, the pacemaker comprises three probes, the third probe being implemented in the coronary sinus. This is particularly advantageous for the treatment or prevention of certain heart failure pathologies.

However, the above examples are not limiting. More generally, any electrical device comprising any number of probes is able to be implemented in the scope of the invention.

According to one embodiment, at least one probe 2 is bipolar. The probe 2 consists of a metal element, for example. The metal element is, for example, a spring. One advantage is that an electrical shock may be delivered by the defibrillator.

In one embodiment, the probe 2 comprises an insulating material. According to one example, the insulating material comprises polyurethane and/or silicone. However, this example is for information only and is not limiting. More generally, the probe 2 is able to comprise any type of insulating material or a combination of several insulating materials together.

According to another aspect, the invention relates to a system configured to implement any one of the steps of the method according to the invention.

In one embodiment, the system comprises the electrical device 1 comprising the probe 2.

In one embodiment, the electrical device 1 comprising the probe 2 is configured to generate the first detection signal Sp. For example, the first S_{D} detection signal is generated in response to the receiving of a cardiac electrical current by the probe 2.

According to one embodiment, the system comprises a memory. The memory is configured to store data. The data includes for example images acquired from the electrogram E_{G} or the item of identification information I_{id} associated with the first detection signal S_{D}. More generally, any type of item of information generated or received by the system is able to be stored in the memory.

In one embodiment, the system comprises a communication interface INT_{C}. In an example, the communication interface INT_{C} is configured to exchange data with at least one other device. According to an example, the communication interface INT_{C} is configured to exchange data with equipment of a data network NET such as a remote server. It should be understood by “exchanging data” that the communication interface “emits” data to remote equipment and that the communication interface INT_{C} “receives” data emitted by remote equipment. According to one example, the data transmitted by the communication interface INT_{C} comprises data stored in the memory of the system. According to another example, the data received by the communication interface is stored in the memory of the system.

According to one aspect, the invention relates to a computer program product comprising instructions which, when the program is executed by a computer, lead the latter to implement any one of the steps of the method according to the invention.

According to one embodiment, the program is loaded onto a medical device comprising a plurality of data recorded in memory. According to one example, the medical device on which the program is loaded comprises an implantable cardioverter defibrillator. In an example, the program implements the steps of the method of the invention from the information recorded in the medical device.

### System

According to another aspect, the invention relates to a system for generating an item of identification information I_{id} of a detection signal.

In one embodiment, the system comprises at least one measuring instrument. In one case, the measuring instrument comprises an instrument for measuring electrical activity; for example, an electrode for measuring cardiac electrical activity.

In one embodiment, the measuring instrument is configured to generate a signal in response to receiving a cardiac electrical current. The generated signal comprises for example a detection signal, such as the first detection signal S_{D}.

In one embodiment, the system comprises a calculator K. According to one case, the calculator K is configured to process data. The processing carried out by the calculator K is implemented, for example, on data measured by means of a measuring instrument, such as a cardiac electrode. In one example, the data processing comprises implementing mathematical operations on said data to be processed, for example comparing values with each other or with threshold values.

In one embodiment, the calculator K is configured to implement an algorithm. The algorithm implemented comprises for example one of the comparison algorithms A_{COMP1}, A_{COMP2}.

In one embodiment, the calculator K is configured to implement one or more functions. The functions implemented by the calculator K comprise for example an assignment function. In an example, the assignment function implemented by the calculator K comprises the assignment of the item of identification information I_{id} to a signal, for example to the first detection signal S_{D}. The item of identification information I_{id} is for example determined based on the result of comparisons performed by one or more algorithms, for example a comparison algorithm A_{COMP1}, A_{COMP2}.

According to one embodiment, the system comprises a display. According to one case, the display is configured to display a graphical representation of a measurement of a physical data or a set of physical data, for example an electrogram E_{G} comprising a detection signal such as the first detection signal S_{D}.

In one embodiment, the display is configured to generate one or more graphical markers overlaying the electrogram E_{G}. The graphical markers include, for example, time markers to bound time intervals associated with the detection signals S_{D}, S_{D-1}, S_{D-2}, S_{D-3}. The time intervals comprise for example the intervals i, i−1 i−2, i−3.

In summary, the invention relates to comparing the time intervals associated with detection signals with threshold values to determine whether these signals are characteristic of a physiological event or whether these signals are characteristic of a hardware defect.

1- A computer implemented method for assigning an item of identification information to a detection signal comprising:
acquiring, by a probe, a first detection signal in response to receiving a cardiac electrical current, said first detection signal comprising a signal portion corresponding to an electrical pattern over a first time interval, said first time interval being defined between two events defined from at least one electrical pattern;
applying a first comparison algorithm comprising:
comparing the first time interval with a first threshold;
comparing a second time interval associated with a portion of a second detection signal prior to the first detection signal with a second threshold;
assigning an item of identification information to the first detection signal, said item of identification information being determined as a function of the result of the comparisons performed by the first comparison algorithm and said item of identification information making it possible to discriminate a detection signal having a physiological cause from a detection signal having for cause a malfunction of electronic hardware.

2- The method according to claim 1, wherein the first comparison algorithm comprises:
comparing a third time interval associated with a portion of a third detection signal with a third threshold, said third detection signal being prior to the second detection signal.

3- The method according to claim 1, comprising:
implementing a second comparison algorithm comprising:
comparing the first time interval with a fourth threshold;
comparing the second time interval with a fifth threshold;
comparing the third time interval with a sixth threshold.

4- The method according to claim 3, comprising implementing, by the first comparison algorithm or the second comparison algorithm, the comparison of a seventh threshold with a fourth time interval associated with a portion of a fourth detection signal prior to the third detection signal.

5- The method according to claim 3, wherein the item of identification information is assigned to the first detection signal according to the result of the comparisons of at least one of the first and second comparison algorithms or a combination of the first and second comparison algorithms.

6- The method according to claim 4, wherein one or more thresholds among the first, second, third, fourth, fifth, sixth and seventh threshold are defined by:
a predefined threshold value or;
a median value or an average of several values together or;
a mathematical function defined relative to one or more time intervals; or
a mathematical function independent of the values of the acquired intervals or;
a combination of several mathematical functions.

7- The method according to claim 4, wherein the item of identification information assigned to the first detection signal comprises either:
an item of physiological information characterizing an item of heart rate data;
an item of anomaly information characterizing an item of non-physiological signal data.

8- The method according to claim 7, wherein the item of identification information assigned to the first detection signal comprises an item of anomaly information characteristic of a probe breakage:
either when the first time interval is less than the first threshold, the second time interval is greater than the second threshold and the third time interval is greater than the third threshold
or when the first time interval is below the first threshold and the second time interval is above the second threshold, the third time interval is above the third threshold and the fourth time interval is above the seventh threshold

9- The method according to claim 7, comprising:
generating a notification or alert when the item of identification information assigned to the first detection signal comprises the item of anomaly information;
saving said alert/notification in a memory space,
emitting said alert/notification generated to equipment of a remote data network.

10- The method according to claim 4, comprising:
generating an electrogram comprising a graphical representation of at least one detection signal among the signals and of at least one time interval;
acquiring, recording and transmitting the electrogram image acquired to at least one piece of equipment in a data network.

11- A system for generating an item of identification information of a cardiac detection signal comprising:
an electrical device comprising at least one probe for acquiring a first detection signal in response to receiving a cardiac electrical current, said detection signal comprising a signal portion corresponding to an electrical pattern over a first time interval;
a calculator configured to implement:
on the one hand, a comparison algorithm comprising:
i. comparing the first time interval with a first threshold;
ii. comparing a second time interval associated with a portion of a second detection signal prior to the first detection signal with a second threshold;
on the other hand, a function for assigning an item of identification information to the first detection signal, said item of identification information being determined according to the result of the comparisons performed by the comparison algorithm, said item of identification information making it possible to discriminate a detection signal having a physiological cause from a detection signal having for cause a malfunction of electronic hardware.
a display for generating a graphical marker overlaid on an electrogram comprising the first detection signal in order to temporally locate the item of identification information of the first detection signal.
a memory for recording data;
a communication interface for exchanging data with equipment in a remote data network;

12- A non-transitory storage medium comprising instructions which, when the instructions are run by a computer, cause the latter to implement the following steps:
a first comparison algorithm comprising:
comparing a first time interval, over which an electrical pattern of a portion of a first detection signal acquired by means of a probe extends, with a first threshold;
comparing a second time interval associated with a portion of a second detection signal prior to the first detection signal with a second threshold;
assigning an item of identification information to the first detection signal said item of identification information being determined as a function of the result of the comparisons performed by the comparison algorithm and said item of identification information making it possible to discriminate a detection signal having a physiological cause from a detection signal having for cause a malfunction of electronic hardware.
